(19) United States
(12) Patent Application Publication
(10) Pub. No.: US 2007/0219632 A1
(43) Pub. Date: Sep. 20, 2007

(54) Method and intra-sclera implant for treatment of glaucoma and presbyopia

(76) Inventor: David Castillejos, La Jolla, CA (US)

Correspondence Address: DONN K. HARMS;PATENT & TRADEMARK LAW CENTER, SUITE 100, 12702 VIA CORTINA, DEL MAR, CA, 92014, US

(21) Appl. No.: 11/801,880

(22) Filed: May 11, 2007

Related U.S. Application Data
(63) Continuation-in-part of application No. 11/528,990, filed on Sep. 27, 2006.
Application No. 11/528,990 is a continuation-in-part of application No. 10/211,197, filed on Aug. 2, 2002.
(60) Provisional application No. 60/800,253, filed on May 12, 2006.
(60) Provisional application No. 60/800,254, filed on May 12, 2006.

(51) Int. Cl. A61F 2/16

(52) U.S. Cl. 623/6.13

(57) ABSTRACT

An apparatus and method for treating presbyopia and lowering intraocular pressure employing an intra-scleral implant into an elongated cavity oriented in the radial direction of the eye. The implant has a planar portion with a longitudinal axis running therethrough and a pair of extension portions extending a distance away from said planar portion and said longitudinal axis. The implant is implanted in an incision in four quadrants of the sclera. The incisions are shaped similar to the implants. The projecting extensions into side projections of the incisions, provide an anchor to maintain the implant in the sclera.

This application is a Continuation in Part of U.S. application Ser. No. 11/528,990 filed Sep. 27, 2006 which is a Continuation in Part of U.S. application Ser. No. 10/211,197 filed Aug. 2, 2002 claiming the benefit of U.S. Provisional Application No. 60/210,227 filed Aug. 3, 2001. This patent application also claims the benefit of the priority of provisional patent application 60/800,253, and claims the benefit of the priority of provisional patent application 60/800,254, filed on May 12, 2006. Each of the non-provisional patent application Ser. Nos. 11/258,990 and 10/211,197, and the provisional patent application 60/210,227, 60/800,253 and 60/800,254, are all incorporated herein by reference.

### BACKGROUND OF INVENTION

1. Field of Invention

The disclosed device relates to a scleral implant. More particularly it relates to a device which is implanted in the sclera within the eye posterior to the limbus, in order to expand the ciliary body and allow the aqueous humor to exit more effectively through the trabecular meshwork (or filtration apparatus) and/or increase the uveal outflow. This surgical placement provides a treatment for excess intraocular pressure which frequently accompanies Glaucoma and for the treatment of presbyopia or loss of accommodation of the eye.

As a continuation to the original application identified above, there are a number of additional modes and embodiments of the disclosed device and method, which can be offered to patients who are candidates for treatments. These include using such implants with or without internal reservoirs and also using an implant as an anchor for attaching a useful reservoir which is engineered to communicate medications to various parts of the eye. Another embodiment describes how such an implant/reservoir complex can be used along with a tube to carry the medication to specifically targeted locations within the eye

Glaucoma is an eye disease wherein the patient gradually loses sight. Such vision loss is caused by damage to the optic nerve which acts like an electric cable and communicates images from the eye to the brain. High intraocular pressure frequently accompanies Glaucoma and is one of the main causes of the nerve damage causing this vision loss. It is thought that increased intraocular pressure is caused when the eye's drainage canals become clogged over time. The intraocular pressure rises to levels causing damage because the correct amount of fluid cannot drain out of the eye in the normal fashion. If this excess intraocular pressure is not detected and treated, it can cause a gradual loss of vision. Such a vision loss in some cases occurs over a long period of time. However, in some cases of glaucoma the eye pressure usually rises very fast. It is thought that this happens when the eye drainage canals are blocked or covered over, like the clog in a sink when something is covering the drain.

Drugs are frequently used on cases where intraocular pressure slowly builds and frequently they work well. In patients suffering a rapid rise in such pressure or a long term rise that has reached a dangerous plateau, severe eye damage and permanent loss of sight can result.

Surgery has also been used more recently to treat intraocular pressure. Clinical investigators have noted in recent years that intraocular pressure is lowered following radial incisions in the anterior sclera, known as an anterior ciliary sclerotomy. Unfortunately, for patients undergoing such a procedure, the beneficial effects are negated over a period of time following the procedure as the incisions heal and scar. Consequently the potential for eyesight loss arises as pressure again builds following the surgery.

Another sight related problem affecting patients is that of presbyopia which is a vision condition in which the crystalline lens of a patient's eye loses its flexibility. This loss of flexibility makes it difficult for a person to focus on close objects. While presbyopia may seem to occur suddenly once the patient discovers the problem, it is generally accepted that the cause of the sight loss is actual loss of flexibility of the lens. This takes place over a number of years and usually becomes noticeable in the early to mid-forties.

Treatment to help you compensate for presbyopia includes prescription reading glasses, bifocals, contact lenses, and laser surgery. However such corrective lenses can be inconvenient to the wearer and laser surgery to the cornea of the eye carries with it the inherent risk to the eyesight itself if a mistake is made.

Still further, many diseases that attack the eye and eyesight require the long term administration of drugs to maintain eyesight. It is desirable to provide an easily placed device that would provide long term modulated direct communication of drugs into the eye concurrently with helping correct the internal pressure and possible vision problems of the patient.

Consequently, there is a continuing need for a medical treatment that would require simple surgical procedure that would have long-lasting effects to relieve internal eye pressure and for the correction of presbyopia to eliminate or reduce the need for prescription lenses and without risky surgery on the lens of the eye itself. Such a treatment would be further enhanced by the provision of a drug delivery system that can be modulated for dose and time that would aid in internal pressure relief as well as other eye ailments requiring precision or long term delivery of drugs.

2. Prior Art

Surgical procedures and implantable devices have recently been developed to address presbyopia. In the past, various ways to design and surgically implant mechanisms that will effectively remove fluid from the eye have been taught. These implementations are typically referred to as “valves”. Such a valve solution will effectively carry unwanted fluids away to a location where such fluid can be either removed or absorbed completely.

U.S. Pat. No. 6,280,468 (Schachar) discloses a scleral prosthesis for treatment of presbyopia and other eye disorders. Schachar teaches the placement of a prosthesis in a plurality of pockets slightly smaller than the implant, circumferentially around the pupil, to exert an outward pressure on the sclera thereby restoring the working distance of the ciliary muscle allowing the patient relief from presbyopia. However, Schachar is oriented circumferentially around the pupil or front of the eye and lacks an anchoring means to hold the implants in proper position in the sclera over the long term which can result in shifting of the implant reducing or eliminating its effectiveness. Further, the use of tunnels smaller than the implant tends to cause broken implants. Schachar also lacks a drug delivery means from the implant. Still further, actual dismounting of the implant can occur which would require removal from the eye especially if it pierces the outside surface of the eye when shifting in position. Additionally, the circumferential placement of the implants is not as effective at encouraging internal drainage and reduction of intraocular pressure.

U.S. Pat. No. 6,102,045 (Nordquist) discloses a method and apparatus for lowering intraocular pressure of the eye. However, Nordquist is a filtering implant which extends into the anterior chamber of the eye through an opening in the limbus cornea. Nordquist lacks the ability to correct presbyopia that a sclera-mounted device provides and because of its delicate positioning and communication directly with the anterior chamber Nordquist is harder to position correctly. It also lacks the ability to infuse drugs to the eye and the provision of direct communication between the anterior chamber and the exterior regions of the eye increases the risk of infection to the anterior chamber.

U.S. Pat. No. 6,079,417 (Fugo) discloses a method and device for reshaping the cornea to change its topography. However, Fugo lacks the ability to increase the drainage from the eye interior to lower intraocular pressure. Fugo also is designed to mount directly into the cornea layer of the eye.

U.S. Pat. No. 5,178,604 (Baerveldt) teaches the use of an implant for increasing eye drainage and reducing pressure caused by glaucoma. However, Baerveldt is simply a tube which communicates directly with the interior chamber of the eye and offers no aid to rectifying presbyopia.

As such, there is a continuing need for a reliable operative method and prosthesis that will aid physicians in interrupting the relentless cycle that results in vision loss and eye damage to patients suffering from building intraocular pressure in the eye. Such a device should be insertable into the eye in a relatively easy procedure for a trained surgeon. Such a device and procedure should avoid the more delicate structures of the eye and should also avoid communicating internal eye structures directly with the exterior of the eye to prevent infection. Such a device would provide additional utility through the optional ability to provide a drug delivery system from the implant directly to the eye. Still further, the device implanted by this method should be dimensioned with an anchor structure to insure that the implant stays properly positioned in perpetuity thereby alleviating the need for replacement or removal caused by dislocatable implants and maintaining a fixed correction of vision.

### SUMMARY OF THE INVENTION

The above problems, and others are overcome by the herein disclosed method and intra-sclera implant for the treatment of glaucoma and presbyopia. As a continuation to the original application identified above, there are a number of additional elements that can be offered to patients who are candidates for treatments using implants with or without reservoirs. These additional applications are presented within this disclosure.

The method of insertion of the implants requires incisions be made radially into the anterior portion of the sclera. A plurality of such incisions are made radially and only into the sclera layer, with the current best number of incisions being four, with one incision within each quadrant of the anterior scleral layer of the eye.

Once the incisions are made in the proper quadrants and extend properly toward the rear of the eye, one implant is positioned within the space of each of the incisions. The scleral incision is then closed by opposition or using suture or other means of closure of the incision to urge the scleral flap toward the surface of the eye from where it was detached and reattach it to the sclera.

The implant is currently best formed in a unitary construction and formed of a material that is inert when in contact with body tissue. Favored materials include one or a combination of materials from a group including hydroxiapartite, silicone, polymethylmethacrylate, acrylic, and tantalum.

The unitary body of the implant can optionally be serrated or have one or a plurality of apertures running through to contact scleral tissue and anchor it. Additionally, the body of the implant can also be impregnated with a drug which thereafter would be slowly delivered into the tissue of the eye or have an internal reservoir or coating of a slowly disbursed drug that can be modulated for dose and time frame to allow for long term delivery of medication to the eye and body of the patient, from the implant.

Additionally disclosed herein are methods and apparatuses surgically placed within the eye, posterior to the limbus, in order to expand the ciliary body and allow the aqueous humor to exit more effectively through the trabecular meshwork (or filtration apparatus) and/or increase the uveal outflow.

In addition to solving problems such as glaucoma and/or presbyopia, other maladies such as macular degeneration and/or diabetic changes and others can be effectively treated. In addition to the aforementioned embodiments there is herein disclosed and described, a number of additional elements which may be offered to patients who are candidates for treatments using implants with or without reservoirs.

As noted above, in one embodiment the device may be employed as a reservoir and hold medication. In another preferred embodiment herein, the reservoir can be a discrete structure which is attached directly to the implant or placed in communication with the implant by way of a conduit or a tube like structure. In this fashion, the implant either functions as an anchor for the reservoir or an ultimate dispensing component for the reservoir. The reservoir itself can be engineered to different dimensional configurations and capacities other than that of the implant if it is placed in fluid communication as a discrete structure. The result being the two entities are combined for maximum effectiveness, however, they are designed and dimensioned with their own purposes in mind. In the end, the implant serves to correct maladies such as increased pressure in the eye and the reservoir serves to hold the highest capacity of medication that can be effectively pumped to the intended part of the eye. Combined, these two novel approaches (the implant and the reservoir) can provide the basis for some of the best possible treatment options for patients suffering from glaucoma and other diseases of the eye. Consequently, an especially preferred embodiment of the device and method herein employs this “combination” approach to maximize the effectiveness of both components to the patient. Such a combined implant and reservoir mechanism is referred to as the implant reservoir complex.

In one preferred embodiment of the combination mode of the invention the implant itself is employed as the anchor mechanism for the reservoir. This allows for a significantly large reservoir to be engaged upon the top of the sclera and underneath the conjunctiva. The reservoir is then connected directly to the implant which serves as an anchor for the reservoir, by way of a small tube or a connecting space. Since it is outside the sclera, such a reservoir can be many orders of magnitude larger than the capacity of the implant itself. Therefore, this design of having the implant function as the “anchor” for the reservoir is novel, useful and important.

A significant problem that arises in all parts of the world is the availability of treatment options and cost. It is common to find people in all parts of the world who either cannot make repeated trips to a physician, or who simply cannot afford the cost of either eye drops or injections (or both). For these patients, such an implant connected directly to a reservoir is extremely valuable. During a routine visit to their physician, they can be outfitted with implants and ample reservoir capacity required for a very long period of time. Therefore, this solution can be used to both reduce the cost of treatment and greatly reduce the frequency that either drops or injections need to be administered to the eye.

Accordingly, it is the object of this invention disclosed herein to provide a reliable method of surgery for the placement of implants in the sclera that is easy to accomplish for the trained surgeon.

It is another object of this invention to provide an implant that is easily insertable into the scleral layer of the eye during a surgical procedure.

It is still another object of this invention to provide such an implant that has an anchoring system to ensure that the implant maintains the position intended by the surgeon implanting it.

Yet another object of this invention is the provision of a method and apparatus for eye surgery that may be used to treat presbyopia as well as rising intraocular pressure.

Still further, it is an object of this invention to provide such an implant with the option of long term drug delivery directly from the implant to the eye.

Yet an additional object of this invention is the provision of medication to the eye rather than simply provide a plumbing mechanism for fluid to drain away from certain areas within the eye.

These and further objectives of this invention will be brought out in the following part of the specification, wherein detailed description is for the purpose of fully disclosing the invention without placing limitations thereon.

### BRIEF DESCRIPTION OF THE DRAWING FIGURES

The accompanying drawings which are incorporated in and form a part of this specification illustrate embodiments of the disclosed device and together with the description, serve to explain the principles of the invention.

FIG. 1 depicts the placement of a plurality of implants radially in four quadrants of the eye and the steps of the method to do so.

FIG. 2 shows the implant and its placement in the scleral layer of the eye.

FIG. 3 depicts a preferred embodiment of the implant showing anchors and optional coating.

FIG. 4 depicts another preferred embodiment of the device having an internal reservoir for holding a drug to be communicated to the exterior.

FIG. 5 depicts another preferred embodiment of the device showing anchors about the exterior.

FIG. 6 depicts another preferred embodiment of the device showing a round body and anchors extending from the surface.

FIG. 7 depicts a particularly preferred mode of the device showing a “Y” or “T” shaped embodiment of the disclosed device implanted in to a similarly formed pocket in the eye.

FIGS. 8-8e are a graphic depiction of the steps of the method of implantation of implants into for quadrants in the eye of a patient.

FIG. 9 of this application shows a very simple diagram that describes how the implant and reservoir complex is communicated to the eye by way of the tube.

FIG. 10 of this application further describes how the implant is being used as an anchor for the reservoir which is intended to provide medication to a specific location within the eye.

FIG. 11 shows how the tube can deliver medication directly into the anterior chamber.

FIG. 12 shows how the tube can be draped around the sclera to reach the posterior pole in close proximity to the optic nerve and the macula.

FIG. 13 shows how the tube can be configured to provide medication through the pars plana into the vitreous cavity.

FIG. 14 shows a configuration where the same tube is performing both the pumping function as well as the drainage function.

FIG. 15 shows how the same tube can be used for both the pumping function and the draining function simultaneously.

FIG. 16 depicts how an implant can be expanded by the injection of an appropriate substance into the implant itself or deflated by draining to a syringe.

FIG. 17 is a two part interlocking device.

FIG. 18 shows the preferred embodiment for such an interlocking mechanism.

### DETAILED DESCRIPTION OF THE PREFERRED EMBODIMENTS OF THE DISCLOSED DEVICE

Referring now to FIGS. 1-18, which in various views depict various preferred embodiments of the disclosed device 10. FIG. 1 depicts the preferred location and steps in the operative method for the placement of the device 10 into the eye 12. The method for surgical insertion of the implant device 10 requires incisions be made radially in the sclera 16 in relation to the cornea 22 and generally in line with the center axis 14 of the eye 12 depicted as running along line 2-2 in FIG. 1.

In its basic structure the eye 12 consists of a globe having an outer coat, a middle layer and an inner layer. The outer coat is made up of a tough fibrous, white layer—the sclera 16, which communicates with the conjunctiva 18 which is a mucous membrane that lines the inner surfaces of the eyelids and folds back to cover the front surface of the eyeball, except for the central clear portion of the outer eye which is the cornea 20. The middle layer contains pigment and forms the iris 22. The inner layer is the light seeing layer or retina 24. The lens 26 is an oval disc which sits behind the iris 22. It is conventional belief that the cornea 20 focuses approximately two-thirds of the light entering the eye 12 and the lens 26 about one third. Lens accommodation or focusing is by simple explanation accomplished by the ciliary muscle 28 pulling upon zonules 30 communicating between the ciliary muscle 28 and the lens 26.

As people age, many suffer from presbyopia which is a vision condition in which the lens 26 loses some of its flexibility, or the zonules 30 become elongated making it harder for the ciliary muscle 28 to focus the lens 26 as needed. Through implanting the device 10 using the surgical method herein disclosed, it is thought that the rejoined sclera 16 tends to pull over the device 10 and impart resulting tension to the ciliary muscle 28 giving it more working distance or travel and resulting ability to pull upon the lens 26 for better accommodation as patients receiving the device using the method of implantation have had improved vision thereafter. It is also thought that a decrease in the lens 26 equatorial diameter and a slight stretching of the zonules 30 increasing their working range also results from the scleral tension developed by the rejoining of the sclera 16 over the inserted device 10, all combine to increase amplitude of accommodation following the implantation surgery.

Intraocular pressure in the eye 12 is caused by a build up of fluid in the anterior chamber 36 and posterior chamber 38 when that fluid which is naturally produced in the eye 12, fails to be communicated through the trabecular meshwork (similar to the grate on a manhole) into the Canal of Schlemm which is the sewer system duct of the eye getting rid of excess fluid and the waste products of the eye. It has been found that following the procedure using the aforementioned method of implantation of the device 10 in the eye of patients that the drainage of aqueous fluid from the eye increases. This results in a drop of intraocular pressure. A tightening of the sclera 16, after implantation of the device 10 in the four quadrants of the eye 12, communicates a tightening or tensioning effect on the ciliary muscle 28 and its connection to the lens 26 and concurrently helps to improve the flow of fluid through the trabecular meshwork in the same region to aid in evacuation of fluid from the anterior chamber 36 and posterior chamber 38. Of course other explanations may be apparent to those skilled in the art and such are anticipated. However in the current best mode patients do experience a drop in fluid pressure in the eye subsequent to the implantation of the device 10 using the method herein disclosed.

In exercising the surgical method for insertion of the implant device 10 the surgeon would begin with a small limited conjunctival peritomy as shown by the conjunctival incisions 32 of FIG. 1. In the current best mode of the method a plurality of conjunctival incisions 32 are performed with four being the current best number, with one in each quadrant of the eye 12 located in-between the muscles 48 attached to the exterior of the eye 12. The conjunctival incisions 32 expose the sclera 16 wherein next, in each conjunctival incision 32, a radial incision 34 is made radially or generally inline with the axis 14 of the eye 12 running through the center of the iris 22 and out the back of the eye 12. The radial incisions 34, it has been found, work well when made posteriorly 0.5 mm from the limbus and measuring substantially 3 mm in length and approximately 600 microns in depth. However, it does depend upon the dimensions of the device 10 implanted and the size of the radial incisions may change to accommodate differently dimensioned devices 10. Such a substantial inline orientation of the radial incisions 34 to the axis 14 or radial to the circle forming the iris 22, has been found to produce the best results for both accommodation and increased drainage of the eye 12.

Once the radial incisions 34 are complete and correctly axially oriented and positioned in the aforementioned manner, an implant device 10 is positioned within the space formed by the radial incision 34. At this point, the radial incision 34 may be closed using a means of closure such as a suture 44 which pulls the scleral flap 21 over the implant device 10 when so rejoined exerting tension upon the sclera 16 and to communicating structures of the sclera 16. Those skilled in the art will recognize that other means of closure of such incisions are available and new means are continually being discovered and the use of such is anticipated. A radial cavity 19 is formed when the scleral flap 21 is rejoined to the sclera which surrounds the implant device 10 was placed in the radial incision. It is also anticipated that the implantation of the implant device 10 radially oriented away from the cornea 20 might be done in other fashions such as drilling or injection or in the future, with a laser or means of mechanization, and such is anticipated. The important aspect of the device and method herein described is that the implant device 10 is placed radially oriented and surrounded by the sclera in a formed cavity and the current best mode of achieving a radial cavity 19 to hold the implant device 10 radially oriented respective to the cornea 20 is by the surgical method herein described.

Following closure of the radial incisions 34, the conjunctival incisions 32 are closed using cautery or other means of closure. The method now being complete, the implant device 10 is properly placed to improve both the vision and fluid drainage of the patient. The implant device 10 may be removed in the reverse order.

The implant device 10 used in combination with the surgical method, in the current best mode, is formed of a material that is inert when in contact with body tissue. The implant device 10 as noted, occupies the radial cavity 19 formed when the radial incision 34 is closed in the aforementioned method. A tightening or tensioning of the sclera 16 layer is provided when the radial incision 34 is closed and the scleral flap 21 is sutured or otherwise rejoined with the sclera 16 and stretched over the implant device 10 during closure. Favored materials include one or a combination of materials from a group including hydroxiapartite, silicone, polymethylmethacrylate, acrylic, and tantalum. Those skilled in the art will recognize that other materials could be used and new materials are continually being developed for implants and the use of such is anticipated.

The implant device 10 has body portion 46 and a means to anchor the device in an elongated cavity oriented in the radial direction of the eye. The cavity, it has been found, as with t all embodiments of the device, works best in combination with the implants when and formed solely within the anterior scleral tissue of the eye cavity 19 to substantially prevent movement, which in a current preferred embodiment is provided by anchors 48 protruding from the body portion 46. Other means to anchor the device when placed in the radial cavity could be accomplished through the use of a serrated surface 50, or curved projections 52, or detents 54 in the exterior surface of the body 46 or apertures 56 which would communicate through the body 46. Or, one or combinations of such means to prevent movement of the implant device 10 can be used together.

Optionally, should the delivery of drugs to the point of implantation be desirable, which with many illnesses such localized delivery is, the device 10 can be provided with a means to communicate drugs from a device resident supply of drugs, to the surrounding eye tissue. This drug delivery system can be provided by one or a combination of micro encapsulated drug coatings or other polymer or prolonged dissolving coatings 58 on the exterior of the device, or through a reservoir 60 inside the body 46 which would hold a supply of the drug of choice in either solid or liquid form and communicate the drugs through channels 62 to the surrounding tissue. Or the material from which the device 10 is produced can be impregnated with the appropriate drug and secrete the same over time. When a reservoir 60 is used, the dosage and delivery time can be modulated by adjusting the amount of communication achieved through the channels 62 or just as the coating can, by adjusting the polymer or other substance in which the drug is dissolved to yield dissolution that will deliver the dose for the amount of time desired for infusion. From the reservoir 60 the device would secrete the drugs over a determined period at the determined dose and then can be refilled through a channel 62 by a hypodermic needle 27 which would pierce the sclera 16 and refill the reservoir 60 through one of the channels 62 or a similar passage designed for such a refill. Refill can thus be accomplished without the need for the implant device 10 to be removed or disturbed from its secure mount inside the radial cavity 19.

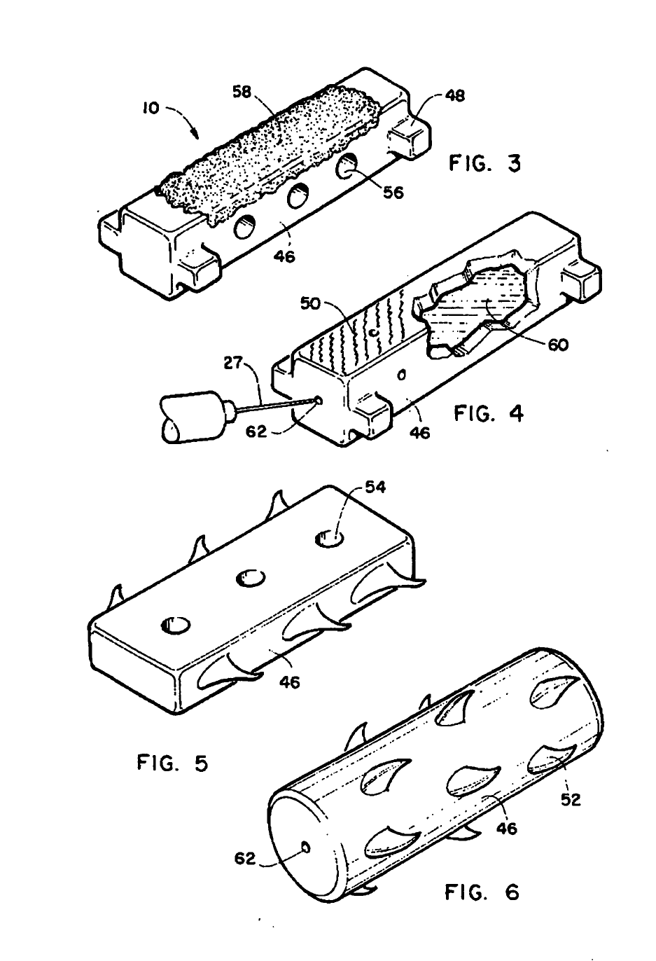

FIG. 6 depicts the device 10 with a body 46 that is round or barrel shaped rather than the cube or rectangular shape of FIGS. 3-5. The body 46 would work well in either configuration so long as one of the noted anchoring means projects from it to anchor the device 10 in the radial cavity. While the curved projections 52 are shown on all sides, it may be beneficial in some cases to omit them from one side for smooth transition of the scleral flap 21 over the implant device 10.

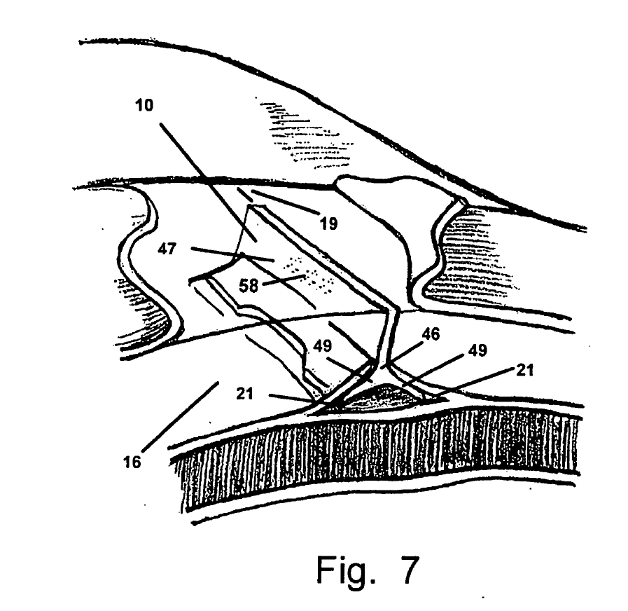

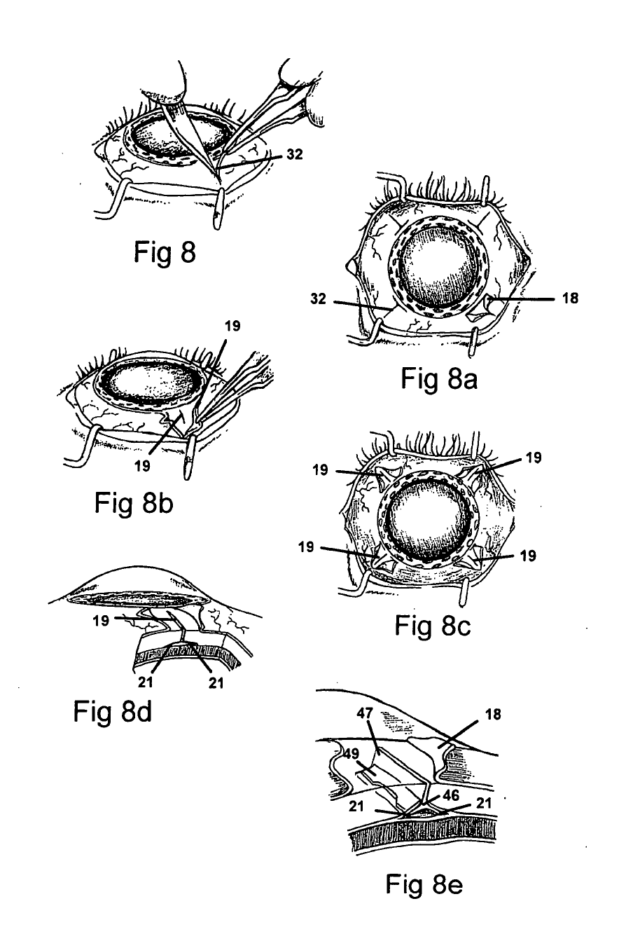

There is depicted in FIG. 7 an especially preferred mode of the device 10 which employs a “T” or “Y” similarly shaped implant device 10 as shown in FIGS. 7 and 8e, which experimentation has shown to be especially effective when implanted into the sclera 16 layer of the eye using a radial incision 34 that is substantially the same shape as the implant device 10. The device 10 as shown in FIG. 7, has a body 46 with a planar component 47 having a first end, and a second end, and a longitudinal axis running through it the same as other embodiments of the device 10. From the second end of the planar component 47, two projecting extension portions, or legs 49, extend away from the axis or plane running through the planar component 47 of the body 46. The two projecting legs 49 have a length substantially equal to that of the planar component 47 and extend a distance from their communication adjacent to the second end of the planar component 47. The result is a “Y” or “T” shaped implant device 10 formed of an elongated body having the planar component 47 and two protecting legs 49. Other shapes and projecting angles and distances could be employed and are anticipated, however the current “Y” or “T” configuration has been shown to be easiest and most accurate for the surgeon to implant by cutting the extensions for the extending legs 49 at the bottom or the radial incision. Further, incisions so formed are predictable in their depth and have increased patient comfort and are considered the favored embodiment of the device because of both considerations.

As shown in FIG. 7, the radial incision 19 situated as noted earlier in a radial orientation of the eye and formed in the preferred mode of the invention solely in the sclera 16. The radial incision 19 has a first portion sized to accommodate the width of the planar component 47 from the first edge closest to the conjunctiva, to the second or lower edge closer to the center of the eye. At a lower edge of the radial incision 19, are formed two side incisions 21 extending from their communication with the radial incision 19 a distance to accommodate the distance dimension of extension of the two legs 49 which is the distance they extend from their respective engagements to the second end of the planar component 47. This results in a substantially “Y” or “T” shaped radial incision 19 formed into the scleral layer of the eye. All the radial incisions 19 are of course oriented in the radial direction of the eye and with their shape or dimension being substantially the same as the implants, provide a mount for the implants therein which will also have their longitudinal axis oriented in the radial direction of the eye once engaged in the radial incision 19.

The two legs 49 extending from their respective engagement with the second end of the planar component 47 provide a means to anchor the device in an elongated radial cavity 19 as they engage with the side incisions 21 along planes which are substantially traverse to the center portion of the radial incision 19. These legs 49 maintain the implant device 10 within the radial cavity 19 in a very secure position. Additionally, it has been found that the legs 49 provide means to impart more tension from the device 10 over a wider area and thereby enhance the resulting tension imparted to the sclera by the device 10 once implanted. This has, as such, enhanced the aforementioned utility of the device 10 to lower intraocular pressure and treat presbyopia. The surface area of the projecting legs 21 and the planar component 47 combine to provide additional, and more even tensioning of the sclera once so implanted, thereby enhancing reduction of intraocular pressure and presbyopia treatment.

FIGS. 8-8e are a graphic depiction of the steps of the method of implant of implants into one or more quadrants in the eye of a patient. First in FIGS. 8 and 8(a) conjunctival incisions 32 are made in the eye in a plurality which as currently noted works best with four. This is filled by the cutting of the radial incision 19 and two side incisions 21 both adapted in depth to accommodate the respective width of the planar component 47 and the distance of extension of the legs 49 from the planar component 47 on the inner edge of the implant device 10. (FIGS. 8b-8d). Finally, the implant device 10 is engaged into the radial incision 19 with the legs 49 engaged into the side incisions 21 and the planar component engaged in the vertical portion of the radial incision 19. Sealing the sclera with sutures or a flap, using this mode of the device 10 with the extending legs 49 is not required since the extending legs 49 provide means to prevent the planar component 47 from translating in the radial incision 19 out of the eye or for that matter, toward the center of the eye. Instead, using the novel extending legs 49 for an anchoring means, the conjunctiva is just rejoined and the device 10 will remain implanted and resist sliding out of the sclera since the two legs 49 are engaged in the side incisions 21 which extend in opposite directions from the central incision holding the planar component 47. Tensioning imparted to the scleral layer over a wider area by both legs 49 and the planar component 47 also yields improved function of the device for both treatment of presbyopia and pressure reduction in the eye. Further, it is much more comfortable for the patient initially and later on with no need for suturing or a scleral flap to hold the device 10 in the mounted position.

As in the other modes of the device 10 noted above, this embodiment with the legs 49 extending from the planar component 47 may optionally be adapted to the delivery of drugs in the same fashion noted above wherein the device 10 is provided with a means to communicate drugs from a device-resident supply of drugs, to the surrounding eye tissue. This drug delivery system can be provided by one or a combination of micro encapsulated drug coatings or other polymer or prolonged dissolving coatings 58 on the exterior of the device as shown in the other figures, or through a reservoir 60 inside the body 46 which would hold a supply of the drug of choice in either solid or liquid form and communicate the drugs through channels 62 to the surrounding tissue. Or the material from which the device 10 is produced can be impregnated with the appropriate drug and secrete the same over time. When a reservoir 60 is used, the dosage and delivery time can be modulated by adjusting the amount of communication achieved through the channels 62 or just as the coating can, by adjusting the polymer or other substance in which the drug is dissolved to yield dissolution that will deliver the dose for amount of time desired for infusion. If a reservoir 60 is employed in the implant 11, it can be refilled by a hypodermic needle 27 which would pierce the sclera 16 and refill the reservoir 60 in a fashion similar to that noted on other embodiments without the need for the implant device 10 to be removed or disturbed from its secure mount inside the radially oriented cavity 19.

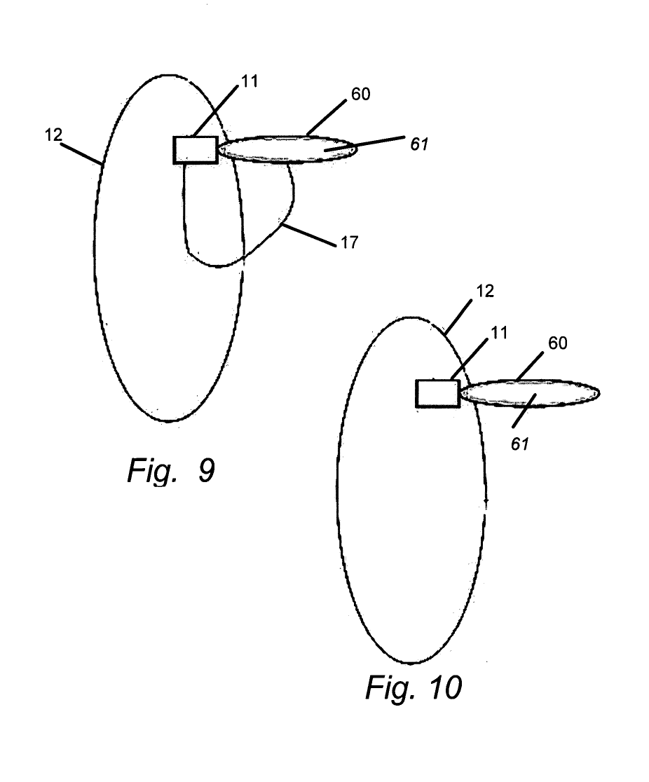

Other modes and particularly preferred embodiments of the device 10 which employ the implant 11 to disburse fluid or as a reservoir 60 and means to disburse fluid to the surrounding tissue are shown in FIGS. 9-18. The embodiments depicted serve to employ an implant 11 engaged with a reservoir 60 or adapted to serve as the reservoir 60 for fluid itself. The range of maladies that can be treated using such a reservoir type implant 11 are various and diverse. It is also important to note that the maladies being treated may not necessarily be related to the eye. For example, it may be possible for the reservoir to deliver medication to people suffering from AIDS in order for them to receive the appropriate and regular dosages (as their bodies have the ability to absorb).

As shown in FIGS. 9 and 10 there are preferred embodiments disclosed and described herein however, employ implants 11 and a reservoir 60 component to treat maladies within the eye. A significant number of maladies relating to the eye need a constant source of medication. Some of these maladies include but are not limited to infections, inflammations, glaucoma, vitreous hemorrhage, various manifestations of diabetes in the eye, various manifestations of vein occlusions in the eye, various proliferative disorders in the eye, various proliferative disorders elsewhere in the body, various ischemic disorders in the eye, macular degeneration, AIDS manifestations in the eye, certain tumors within the eye, certain tumors in other parts of the body, pain management, and other maladies adapted for treatment with a constant flow of medication.

A new concern being considered by opthalmologists at the time of this writing is that of VEGF, or vaso-endothelial growth factor. Such an implant containing anti-VEGF therapy along with a reservoir connected to it can greatly reduce the problems introduced by the need for frequent and repeated injections into the eye.

Not only Anti-VEGF therapy but other therapies require the need for frequent and repeated applications of medication to specific locations within the eye. In some cases these applications may be provided by the use of eye drops. In other cases, these applications may require an injection into the eye.

In a first preferred method of employment of the implant 11 and reservoir 60 combination herein, a qualified surgeon such as a licensed opthalmologist performs a surgical procedure including the steps of:

A. The surgeon would make a conjunctival incision 32 in the conjunctiva 33 in order to expose the selected area of the sclera 16 where the implant 11 will be placed.

B. The site of the placement is marked.

C. A sclera incision 19 is performed radially starting 0.5 millimeters posterior to the limbus 23 in the selected quadrant and continuing the incision for a total of 3 millimeters. The appropriate depth of the incision is achieved when the blue hue of the choroid can be visualized by the surgeon.

D. Side incisions 21 or pocket incisions are then performed on both sides of the base of the incision 19, i.e., the portion closest to the choroid.

E. An implant 11 is then placed in that incision 19 with the foot-plates of the implant trapped within the side incisions 21 which form the pockets.

F. If the implant 11 is being used along with a reservoir 60, then the reservoir 60 is placed posterior to it and preferably either at the level of the equator or posterior to such a landmark.

G. An optional 10-0 nylon suture can be used to approximate the incision 19 to further decrease the possibility of implant extrusion.

H. The conjunctiva 33 and the Tenon's capsule are closed on top of the implant and the reservoir 60 to assure their protection.

I. The patient would be sent to recovery.

The patient will need the administration of antibiotic and anti-inflammatory eye drops and/or ointments for a short period of time. Typically the time period would be one to three weeks. The typical period of recovery will also be from one to three weeks. However, after four or five days, the patient will be able to resume normal activities. It is typical for the patient to experience some redness and mild discomfort during the recovery period. Certain painkillers may be used by the patient during the first few days after the procedure.

Treatments involving such an implant/reservoir complex as described in this application will therefor allow for the constant and slow release (i.e., a constant rate of delivery) of medication for very long period of time. Furthermore, the reservoir can be refilled offering ongoing therapy without the need of injections into the internal cavities of the eye or the use of eye drops.

In regards to the results of such treatments, the medication will be absorbed by the intended tissue over the course of time. The process of the medication being absorbed will effectively “pull” the new medication from the reservoir to the intended tissue by way of the tube.

In a preferred embodiment, shown in FIG. 10, wherein the all of the component parts of the combined implant 11 and reservoir 60 complex (the implant 11, the reservoir 60, and the tube 17) will be in a relatively close proximity to the intended tissue, however, as long as the tube 17 can deliver the medication to the intended tissue effectively, the various component parts do not need to be in such close proximity. The reservoir 60 should be formed to be generally planar some what like a hollow pancake, where sidewalls surround an internal cavity 61 of the reservoir 60 and so as to conform to the exterior surface of the eye when anchored there. The internal cavity of the reservoir holds the medicine to be communicated through the tube 17 to the interior cavity of the implant 11.

Once the recovery period is over and the patient no longer experiences any discomfort, the constant and steady delivery of medication from the internal cavity 61 of the reservoir 60 through the interior cavity of the implant 11, to the body, should greatly reduce the patient's symptoms. The tube 17 will effectively provide an adequate amount of medication to the intended tissue for a time frame that can be predetermined by the surgeon. This process of providing the constant and steady delivery of the intended medication is called “determinate pumping.”

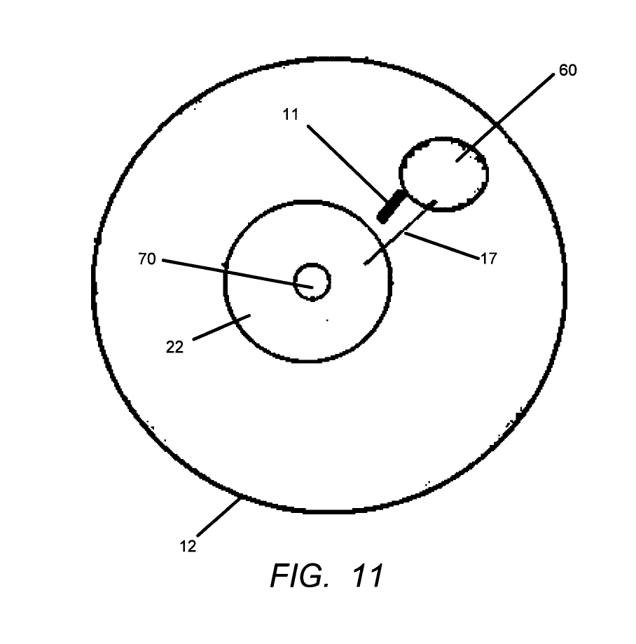

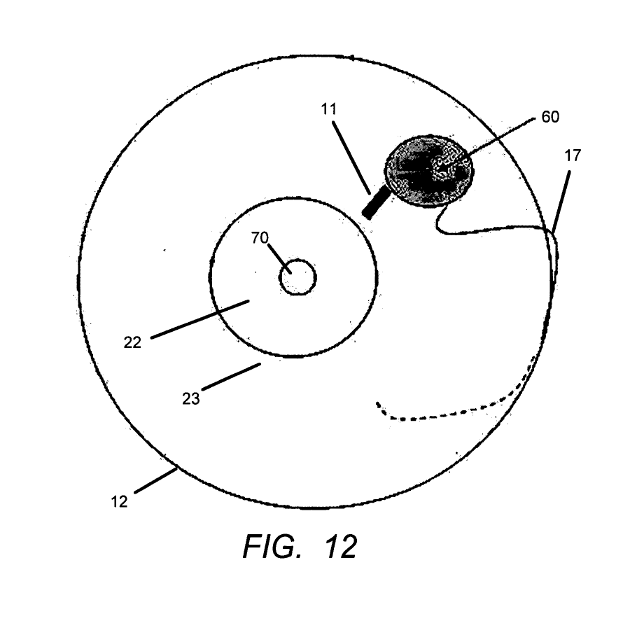

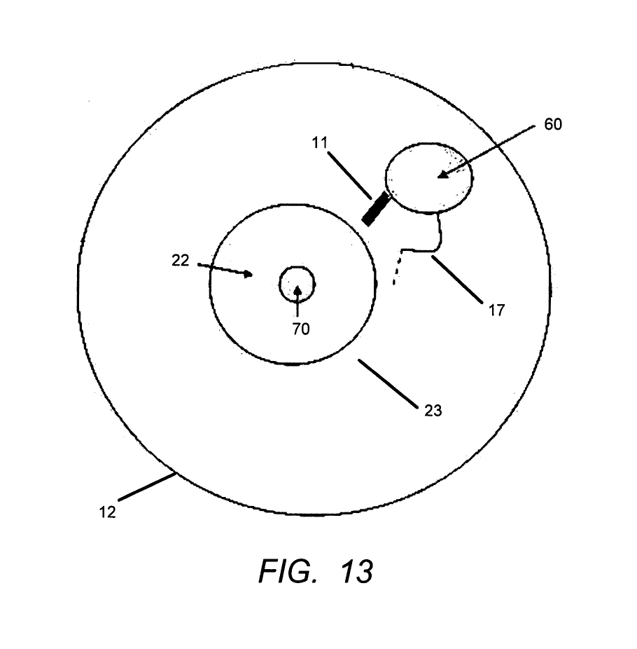

FIGS. 11-13 of this application show the various ways in which the tube 17 can be configured to deliver the medication to the intended location within the eye 12. FIG. 11 shows how the tube 17 can deliver medication from the internal cavity of the pancake sytle reservoir 60 directly into the anterior chamber 36. FIG. 12 shows how the tube 17 can be draped around the sclera 16 to reach the posterior pole (in close proximity to the optic nerve 72 and the macula). FIG. 13 shows how the tube 17 can be configured to provide medication through the pars plana into the vitreous cavity. It is important to note that virtually any arrangement of the tube 17 can be configured in order to deliver medication from the reservoir 60 which is adapted in its planar or curved planar shape for engagement adjacent to the exterior hemisphere of the eye 12 to the intended parts of the eye 12.

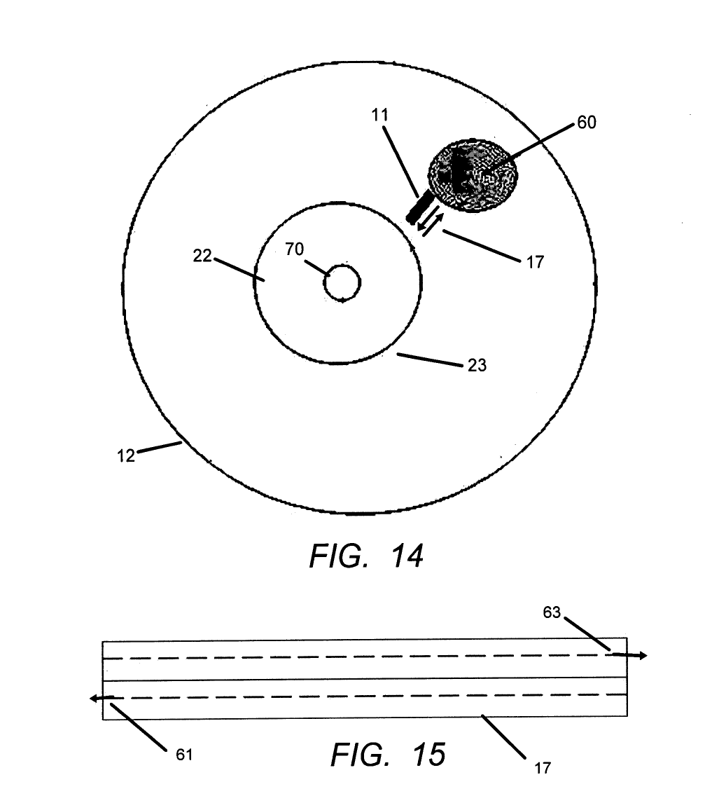

In FIGS. 14-15 there is shown another preferred mode of the device and method herein disclosed, where there can be alternatively provided both a “determinate pumping” function in addition to a simultaneous draining function. Therefore, the process of deliberately pumping medication and draining the eye of unwanted fluids can take place simultaneously.

FIG. 14 shows a configuration where the same tube 17, having double conduits, is performing both the pumping function as well as the drainage function. The medication 61 is being pumped from the reservoir 60 into the anterior chamber 36 and unwanted fluids 63 are being drained from the anterior chamber 36 into the Reservoir 60. Unwanted fluids 63 will then have a chance to be absorbed and/or otherwise removed completely. FIG. 15 depicts how the same tube can be used for both the pumping function and the draining function simultaneously.

Reservoir—Semipermeable Membrane

The reservoir 60 itself or the implant 11 itself may also be employed as a delivery system with a semipermeable membrane which would require manufacturing the reservoir 60 (or the implant/reservoir complex) to contain a semipermeable membrane on at least one of its walls. Such material is well known in the art and those skilled in the art will realize that various types may be employed herein. This will allow diffusion of medication from the interior of either the implant 11 or the reservoir 60 or both, toward the exterior environment which will then allow the absorption into the eye.

Reservoir—Biodegradable

Another preferred mode of the device provides for the implant 11 to be manufactured using virtually any biodegradable material compatible and nontoxic to the eye. Such a material will be designed to store the intended medication and then slowly degrade after or while the medication is consumed. The implant 11 would therefor be absorbed in the body of the patient over time and no surgery would be required for removal. In such a preferred embodiment, the process of the material being degraded (over time) will cause the fluid to be “pumped” to the intended location in the eye 16. Therefore, the material will be completely degraded at virtually the same time as the remainder of the medication is disbursed.

As noted, a particularly preferred mode of the device provides that the implant 11 and reservoir 60 complex may be manufactured with a membrane (at one or more of its walls) that will allow repeated injections of the same or a different medication into the implant 11 in such a way that allows the refilling of the reservoir 60 cavity for a potentially long and undetermined length of time—possibly years, decades, or even longer.

It has been also noted that preferred numbers of implants 11 is one to four implants 11 will be used in each eye. However, it is possible to populate each eye with more implants depending upon the desired result. Likewise, numerous discrete reservoirs 60 can be populated within each eye. The implants 11, reservoirs 60, and implant/reservoir complex units, can be engineered in many different ways in order to achieve numerous desired effects.

It is not necessary that only one implant 11 (used as an anchor) be coupled with only one reservoir 60. In fact, the number of implants 11 is virtual and the number of reservoirs 60 is virtual in the system disclosed herein. Therefore, there can be two implants 11 for one reservoir 60 and there can be two reservoirs 60 for one implant 11. The number of implants 11 and/or reservoirs 60 is virtual depending on the needs of the patient.

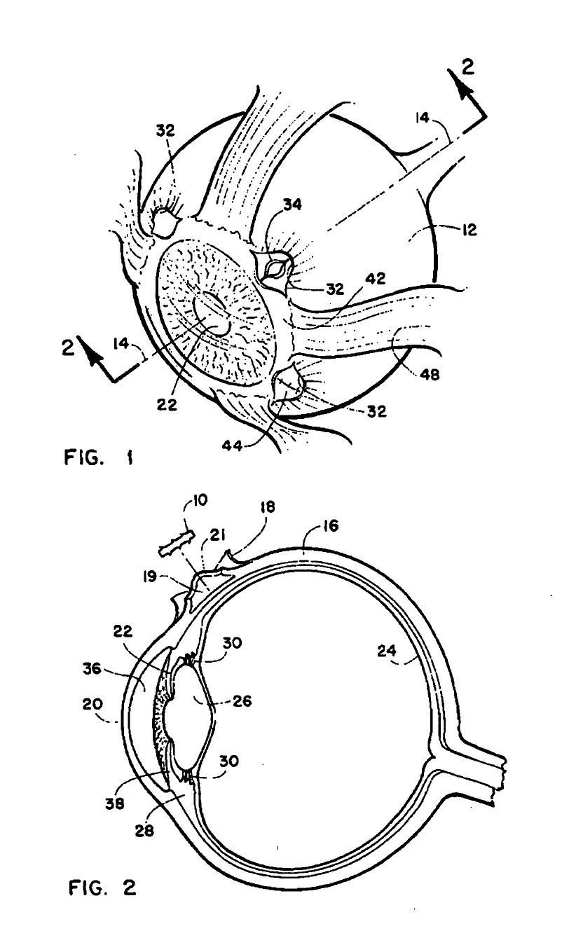

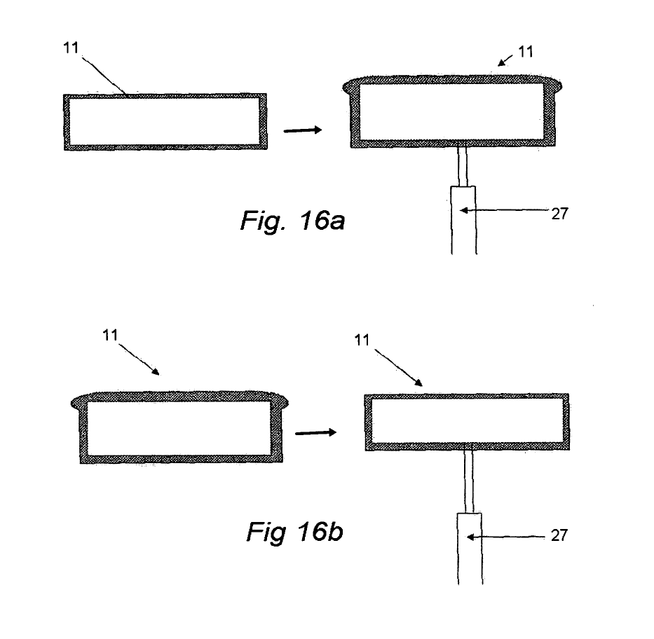

In FIG. 16 there is depicted an implant 11 which would be implanted as in FIGS. 1-2 and has flexible sidewalls forming the implant 11 and defining an internal chamber in the implant 11. With the flexible walls the implant 11 can be expanded by the injection of an appropriate substance into the implant 11 or deflated by draining to a syringe 27. This embodiment would be most effective as the amount of tension imparted to the sclera 16 can be varied by increasing or decreasing the overall dimension of the flexible implant 11.

FIG. 17 shows an embodiment of the device which features an interlocking two part tube 17 which would allow for engagement and disengagement of the reservoir 60 to the implant 11. A tube coupling device and interlocking device conventionally employed to join tubing 17 in medical devices would be employed and would allow for easy changing of the reservoir 60 to change the fluid supplied or to refill the fluid being supplied.

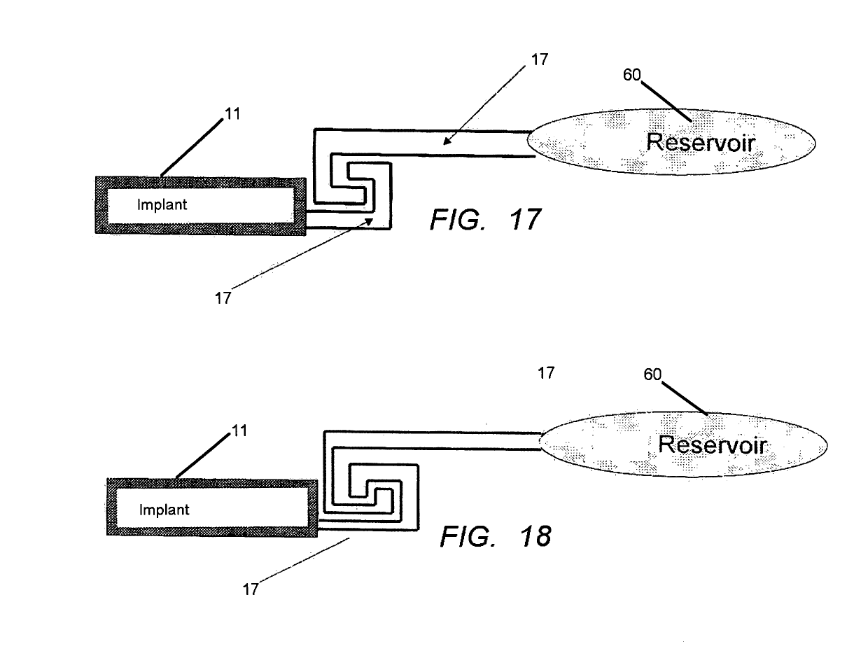

FIG. 18 shows the preferred embodiment similar to that of FIG. 17 however the interlocking mechanism for the tube 17 portions has at least three angles within the interlocking mechanism. The resulting overhang of each distal end of each section of tube 17 ensures that the tubing will not come apart if jarred or otherwise pulled upon since both distal ends to the two tubing sections when engaged are surrounded by two wall portions of the tubing 17 of the other half of the pair of tube sections forming the tube 17 when joined. This type of engagement provides a sealed communication of fluid through the tubing sections, and prevention from lateral forces unhooking the sections.

The device and method herein described and disclosed provides a novel delivery system that will allow treatment and supportive therapy for a wide number of eye (and other systemic) disorders. Specifically, it is the combination of the implant coupled with a reservoir (called the implant/reservoir complex) that offers viable treatment options and remedies to patients around the world. While all of the fundamental characteristics and features of the present invention have been described herein, with reference to particular embodiments thereof, a latitude of modification, various changes and substitutions are intended in the foregoing disclosure and it will be apparent that in some instances, some features of the invention will be employed without a corresponding use of other features without departing from the scope of the invention as set forth. It should be understood that such substitutions, modifications, and variations may be made by those skilled in the art without departing from the spirit or scope of the invention. The range of maladies that can be treated using such a reservoir are various and diverse. It is also important to note that the maladies being treated may not necessarily be related to the eye. For example, it may be possible for the reservoir to deliver medication to people suffering from AIDS in order for them to receive the appropriate and regular dosages (as their bodies have the ability to absorb). Consequently, all such modifications and variations are included within the scope of the invention as defined by the following claims.

What is claimed is:

1. An intra-scleral implant for delivery of medication to a patient, comprising:
an elongated body dimensioned for implantation into an elongated cavity in the anterior scleral tissue of the eye;
said body having an internal cavity;
means for communication between said internal cavity and an area adjacent to said body;
a reservoir component having an interior cavity adapted for storage of a medication;
means for engagement of said reservoir component to the exterior hemisphere of the eye; and
means for sealed communication between said internal cavity and said interior cavity, whereby said medication from said reservoir is communicated to said implant for administration to a patient in which said implant is placed.

2. The intra-scleral implant of claim 1 additionally comprising:
said elongated body engaged in said cavity having a direct connection to said reservoir in an engaged position; and
said body providing a means to anchor said reservoir to said eye when in said engaged position.

3. The intra-scleral implant of claim 1 wherein said means for sealed communication between said internal cavity and said interior cavity comprises:
a flexible tube having an axial passage in sealed communication with said internal cavity and said interior cavity.

4. The intra-scleral implant of claim 2 wherein said means for sealed communication between said internal cavity and said interior cavity comprises:
a flexible tube having an axial passage in sealed communication with said internal cavity and said interior cavity of said reservoir.

5. The intra-scleral implant of claim 3 additionally comprising:
said flexible tube having a first tube component extending from a sealed communication with said internal cavity to a first distal end;
said flexible tube having a second tube component extending from a sealed communication with said interior cavity, to a second distal end;
means for removable sealed engagement of said first distal end to said second distal end, whereby said reservoir is removably engageable with said body of said implant to provide a means to refill said internal cavity with medicine from subsequent said reservoir components having a said second tube component engageable with said first tube component.

6. The intra-scleral implant of claim 4 additionally comprising:
said flexible tube having a first tube component extending from a sealed communication with said internal cavity to a first distal end;
said flexible tube having a second tube component extending from a sealed communication with said interior cavity, to a second distal end;
means for removable sealed engagement of said first distal end to said second distal end, whereby said reservoir is removably engageable with said body of said implant to provide a means to refill said internal cavity with medicine from subsequent said reservoir components having a said second tube component engageable with said first tube component.

7. The intra-scleral implant of claim 1 additionally comprising:
said elongated body having a planar portion have a first end, a second end;
a first side and a second side of said planar portion communicating between said first and second end;
said elongated body having a longitudinal axis running between said first end and said second end, which longitudinal axis, when implanted, is oriented in the radial direction of the eye; and
a pair of extension portions each extending a substantially equal distance away from said planar portion and said longitudinal axis;
said planar portion and said extension portions forming said implant in a substantially “T” shape when viewed from said first or said second end;
said extension portions positioned for an engagement with side cavity portions extending from said elongated cavity in said sclera; and
said extension portions when in said engagement thereby providing means to anchor said implant in said cavity.

8. The intra-scleral implant of claim 1 wherein said means for communication between said internal cavity and an area adjacent to said body is a semipermeable membrane.

9. The intra-scleral implant of claim 7 wherein said means for communication between said internal cavity and an area adjacent to said body is a semipermeable membrane.

10. The intra-scleral implant of claim 1 wherein said elongated body is formed of biodegradable material absorbable by the body of a patient in which said implant is placed.

11. The intra-scleral implant of claim 7 wherein said elongated body is formed of biodegradable material absorbable by the body of a patient in which said implant is placed.

12. An intra-scleral implant for delivery of medication to a patient, comprising:
an elongated body dimensioned for implantation into an elongated cavity in the anterior scleral tissue of the eye;
a reservoir component having an interior cavity adapted for storage of a medication;
means for engagement of said reservoir component to said body wherein said body mounted in said elongated cavity provides a means to anchor said reservoir component to said eye; and
an elongated flexible tube having a first end in sealed communication with said interior cavity; and
said elongated flexible tube having a second end engageable to a position in the interior of said eye whereby said medication from said reservoir is communicated to eye tissue at said position in the interior of said eye of a patient in which said implant is engaged.

13. The intra-scleral implant of claim 12 additionally comprising:
said flexible tube having a second axial passage in sealed communication with said internal cavity; and
said second axial passage providing means to drain fluid from said eye tissue to said interior cavity.

14. The intra-scleral implant of claim 12 additionally comprising:
said elongated body having a planar portion have a first end, a second end;
a first side and a second side of said planar portion communicating between said first and second end;
said elongated body having a longitudinal axis running between said first end and said second end;
a pair of extension portions each extending a substantially equal distance away from said planar portion and said longitudinal axis;
said extension portions positioned for an engagement with side cavity portions extending from said elongated cavity in said sclera; and
said extension portions when in said engagement thereby providing means to anchor said implant in said cavity.

15. The intra-scleral implant of claim 1 additionally comprising:
said elongated body formed of flexible material whereby said body increases in dimension when said internal cavity is filled.
